(19) United States
(12) Patent Application Publication
(10) Pub. No.: US 2022/0196653 A1
(43) Pub. Date: Jun. 23, 2022

(54) METHOD FOR DETECTING OR MONITORING THE PROGRESSION OF A CHRONIC AUTOIMMUNE DISEASE BY IMMUNOASSAY

(71) Applicant: POLYNEUROS, SAINT JEAN D'ILLAC (FR)

(72) Inventors: Michel GEFFARD, Talence (FR); Jean-Pascal ZAMBAUX, Bouliac (FR)

(21) Appl. No.: 17/600,743

(22) PCT Filed: Apr. 1, 2020
(86) PCT No.: PCT/EP2020/059212

§ 371 Date: Oct. 1, 2021

(30) Foreign Application Priority Data
Apr. 1, 2019 (FR) FR1903453

(51) Int. Cl. G01N 33/564; G01N 33/569

(52) U.S. Cl. CPC G01N 33/564; G01N 2800/285; G01N 33/5695

(57) ABSTRACT

The invention relates to an ex vivo method for detecting or monitoring the progression of a chronic autoimmune disease, in a sample of human or animal biological fluid, by immunoassay for the presence of antibodies in the sample, including at least:
one or more antibodies directed against at least one enterobacterium, and
one or more antibodies directed against a product at the origin of or resulting from lipoperoxidation and/or one or more antibodies directed against a nitrated or nitrosylated product.

The invention also relates to a kit for implementing such a method.

### CROSS-REFERENCE TO RELATED APPLICATIONS

This application is a U.S. National Stage Application of PCT/EP2020/059212 assigned the international filing date of Apr. 1, 2020 and claiming the benefit of priority from EP patent application FR1903453 filed Apr. 1, 2019, the disclosure of these applications is herein incorporated by reference.

### TECHNICAL FIELD

The invention relates to the ex vivo detection and monitoring of the progression of chronic autoimmune diseases in humans or animals.

### BACKGROUND

Autoimmune diseases are pathologies that result from a dysfunction of the immune system, leading it to attack the components of the body.

In addition to having the characteristic of being autoimmune, proliferative and/or degenerative processes can develop during the progression of the disease. This is called comorbidity, which corresponds to the result of the association between autoimmune disease and degenerative and/or proliferative disease.

The therapeutic solutions provided depend on the type of autoimmune disease and often involve drugs that suppress the activity of the immune system. These drugs also have very limited or no effectiveness, are generally toxic and have significant side effects.

In addition, treatments are often ineffective in the very frequent cases where autoimmune diseases are diagnosed too late to be able to stop or slow the progression of the disease in time. In fact, there is no satisfactory diagnostic test. Several blood tests are usually done to look for a possible autoimmune disease. It is generally not known what triggers autoimmune diseases, and symptoms vary depending on the disorder that develops and the part of the body that is affected.

In addition, since these pathologies are chronic, they require regular monitoring of the progression of the disease, and there is currently no simple and reliable solution that allows such monitoring.

### SUMMARY

The objective of the invention is to overcome the drawbacks of the prior art by proposing a method for both detecting and monitoring the progression of a chronic autoimmune disease in humans or animals, which is simple to implement, minimally invasive, fast, efficient and reliable.

To this end, the object of the invention is an ex vivo method for detecting or monitoring the progression of a chronic autoimmune disease, in a sample of human or animal biological fluid, preferably of human or animal plasma and/or serum, by immunoassay for the presence of antibodies in the sample, including at least:
one or more antibodies directed against at least one enterobacterium, that is to say, against at least one antigenic component of at least one enterobacterium, and
one or more antibodies directed against a product at the origin of or resulting from lipoperoxidation, and/or
one or more antibodies directed against a nitrated or nitrosylated product.

The method can also comprise the immunoassay for the presence in the sample of:
at least one antibody directed against a tryptophan oxidation product, and/or
at least one antibody directed against a nitrated or nitrosylated product, and/or
at least one antibody directed against Mycobacterium tuberculosis.

Advantageously, this method is simple to implement, since it is carried out on a sample of biological fluid and it implements conventional immunoassay techniques. The immunoassay method mimics ex vivo what happens in vivo where antigens are bound to immunoglobulins (immune complexes).

The method according to the invention can be implemented using a kit that constitutes another object of the invention.

Other features and advantages of the invention will emerge from the detailed description that follows.

### DESCRIPTION OF THE FIGURES

FIG. 1 shows the results of the detection of circulating antibodies directed against quinaldic acid in IgA, IgM and IgG, picolinic acid in IgA, IgM and IgG and anthranilic acid in IgA, IgM and IgG, during the progression of multiple sclerosis, as a distribution of a population of multiple sclerosis patients; the midpoint represents 50% of the population.

FIG. 2 shows the results of the detection of circulating antibodies directed against Mycobacter tuberculosis in IgA, IgM and IgG, during the progression of multiple sclerosis, as a distribution of a population of multiple sclerosis patients; the midpoint represents 50% of the population.

FIG. 3 shows the results of the detection of circulating antibodies directed against NO-cysteine in IgA, IgM and IgG, NO-tryptophan in IgA, IgM and IgG and NO-asparagine in IgA, IgM and IgG, during the progression of progressive multiple sclerosis, as a distribution of a population of multiple sclerosis patients; the midpoint represents 50% of the population.

FIG. 4 shows the results of the detection of circulating antibodies directed against NO-citrulline in IgA, IgM and IgG, NO-histidine in IgA, IgM and IgG and NO-methionine in IgA, IgM and IgG, during the progression of progressive multiple sclerosis, as a distribution of a population of multiple sclerosis patients; the midpoint represents 50% of the population.

FIG. 5 shows the results of the detection of circulating antibodies directed against NO-tyrosine in IgA, IgM and IgG, NO2-tyrosine in IgA, IgM and IgG and NO-phenylalanine in IgA, IgM and IgG, during the progression of relapsing-remitting multiple sclerosis, as a distribution of a population of multiple sclerosis patients; the midpoint represents 50% of the population.

FIG. 6 shows the results of the detection of circulating antibodies directed against NO-histidine in IgA, IgM and IgG, NO-arginine in IgA, IgM and IgG and NO-BSA in IgA, IgM and IgG, during the progression of relapsing-remitting multiple sclerosis, as a distribution of a population of multiple sclerosis patients; the midpoint represents 50% of the population.

FIG. 7 shows the results of the detection of circulating antibodies directed against NO2-tyrosine in IgA, IgM and IgG, NO-phenylalanine in IgA, IgM and IgG and NO-BSA in IgA, IgM and IgG, during the progression of multiple sclerosis, as a distribution of a population of patients with a progressive form of multiple sclerosis versus a relapsing-remitting form; the midpoint represents 50% of the population.

FIG. 8 shows the results of the detection of circulating antibodies directed against NO-cysteine in IgA, IgM and IgG, NO-tryptophan in IgA, IgM and IgG and NO-asparagine in IgA, IgM and IgG, during the progression of multiple sclerosis, as a distribution of a population of patients with a progressive form of multiple sclerosis versus a relapsing-remitting form; the midpoint represents 50% of the population.

FIG. 9 shows the results of the detection of circulating antibodies directed against NO-creatinine in IgA, IgM and IgG, NO-tyrosine in IgA, IgM and IgG and NO-arginine in IgA, IgM and IgG, during the progression of multiple sclerosis, as a distribution of a population of patients with a progressive form of multiple sclerosis versus a relapsing-remitting form; the midpoint represents 50% of the population.

### DEFINITIONS

Within the meaning of the invention, “circulating antibodies” refers to antibodies that are found in human or animal biological fluids, in particular in the blood, serum and/or plasma of humans or animals.

Within the meaning of the invention, “reagent blank” refers to a solution or a mixture containing all the reagents used during the method according to the invention, but which does not contain the sample to be analyzed. It allows a basic correction to be made to the results of the assay. It defines the background noise of the assay method.

Within the meaning of the invention, “biological fluid” refers to fluid from the body of a human being or of an animal, in particular blood, serum and/or plasma, cerebrospinal fluid, pleural fluids and intraperitoneal, intra-articular fluids, saliva or urine.

Within the meaning of the invention, “neoantigen” refers to the result of a covalent bond between a radical compound (NO, NO₂, etc.) or at the origin of or resulting from lipoperoxidation (Malondialdehyde, Azelaic acid, etc.) or resulting from the hyperproduction of metabolic compounds (derivatives of tryptophan), and an endogenous cellular or tissue component. A neoantigen is also the result of unmasking a cellular component (fatty acid, phosphatidylinositol, etc.) not physiologically accessible to the immune system that, following active inflammatory processes, is exposed to the immune system.

Within the meaning of the invention, “lipoperoxidation product” or “product associated with lipoperoxidation mechanisms” refers to a product that is at the origin of or results from the peroxidation of lipids, i.e. the oxidation of unsaturated lipids, either by radical species of oxygen and nitrogen, or catalyzed by enzymes. The peroxidation of lipids results in their degradation into products called lipoperoxidation products.

Within the meaning of the invention, “nitrated or nitrosylated product” refer to neoantigens resulting from an overproduction of NO and/or peroxynitrite. This hyperproduction results from the activation of inducible NO synthase, mainly in the cells of the innate immune system, by bacteria, viruses, pollutants, etc.

Within the meaning of the invention, the term “tryptophan oxidation product” refers to a metabolite resulting from the degradation of tryptophan, after the enzymatic activation of the Indolamine-2,3-Dioxygenase (IDO)-1 and/or THO (Tryptophan hydroxylase) pathways. This enzymatic activation takes place in mono-macrophagic cells under the action of bacterial, viral, mycotic, pollutant, and/or pro-inflammatory cytokine and chemokine components. “Bacterial component” refers to a bacteria degradation product that results from bacterial mechanical or biochemical lysis.

### DETAILED DESCRIPTION OF THE INVENTION

The object of the invention is therefore an ex vivo method for detecting or monitoring the progression of a chronic autoimmune disease.

The disease can be any chronic autoimmune disease, and in particular it can be a disease selected from multiple sclerosis, rheumatoid arthritis and ankylosing spondylitis.

The method is carried out ex vivo, outside the human or animal body, in a sample of human or animal biological fluid, preferably of human or animal serum or plasma, which was taken before the implementation of the method. The sample of biological fluid is a sample that has been taken and stored according to the usual standards known to those skilled in the art.

The method according to the invention is carried out by immunoassay for the presence in the sample of circulating antibodies directed against specific antigens of chronic autoimmune diseases.

The method can be any type of immunoassay method. It may in particular be an ELISA assay, strip tests, tests carried out using magnetic, latex and/or silica beads, or else a Western blot test. These tests can be implemented according to the knowledge of a person skilled in the art.

In the implementation of the method according to the invention:
The antigens and neoantigens used to implement the method are preferably antigens synthesized ex vivo. In the case where the antigens are components of bacteria, they can be obtained from bacterial cultures by sonication and/or in the presence of enzymes (Lysozyme) and anti-proteases (Pepstatin, Aprotinin etc.),
If the antigens used are not bacterial components, they are preferably coupled (conjugated) to a protein, such as Bovine Serum Albumin for example, to facilitate the attachment of the antigen to the support (plate, strip, etc. depending on the assay method); thus, an antibody directed against a neoantigen within the meaning of the present invention can mean (excluding bacterial antigens) an antibody directed against a conjugated antigen, whether or not the expression “conjugated” is indicated after the neoantigen.
The secondary antibodies, called anti-isotypes, conjugated to an enzyme such as peroxidase or alkaline phosphatase, are diluted according to their isotypy, in a diluting buffer, called preservation buffer, containing stabilizing proteins. These antibodies are preferably known antibodies that can be purchased.

The method according to the invention preferably comprises an enzyme immunoassay based on the ELISA method. The immunoassay method can comprise the following steps:
the antigens against which the antibodies to be detected in the sample are directed are adsorbed (and optionally dried) on microtiter plates; this step consists in sensitizing microtiter plates with the antigen(s) against which the antibodies, the presence of which is to be detected in the sample, are directed.

The plates are then either used directly to implement the method, or dried and stored in a dry atmosphere, preferably at 4° C. and protected from light.
the sample to be tested is then preferably diluted; this dilution can be between 125 and 1000 times, in particular 150 to 1000 times;
similarly, preferably, test sera or plasmas, called internal standards, which are biological fluids from healthy subjects, preferably sera or plasma from healthy subjects (representative population matched with the patient population at least for sex and age), are used and preferably diluted; this dilution can be between 125 and 1000 times, in particular 150 to 1000 times;
the sample, a reagent blank and preferably also the internal standards, are distributed, preferably in duplicate, in wells of at least one sensitized microtiter plate,
preferably, the plate(s) are incubated; according to a particularly suitable embodiment, they are incubated between 1 h 30 and 2 h 00, preferably between 1 h 45 and 2 h 00, at a temperature of 35 to 39° C.,
preferably, the plate(s) are washed, that is to say, rinsed, preferably several times, with the aim of eliminating all the non-specific proteins and immunoglobulins;
then, anti-human or animal immunoglobulin antibodies, called secondary antibodies, are added that are directed against isotypes A, M or G for humans, A, M, G or E in animals; these secondary antibodies are antibodies directed against the immunoglobulins bound to the antigens present in the wells of the microtiter plates; the secondary antibodies are preferably coupled to an enzyme, and preferably to alkaline peroxidase or phosphatase, to allow a colorimetric reaction to be carried out,
preferably, the plates are incubated again for 1 hour to 2 hours at a temperature between 35 and 39° C.,
preferably, the plates are then washed again, that is to say, several rinses are carried out so as to eliminate what has not been specifically recognized,
preferably, a substrate of the enzyme (H₂0₂ for peroxidase or for alkaline phosphatase) is then applied with a chromogen; the objective of this step is to visualize the immunological reactions. The chromogen can for example be tetramethylbenzidine (TMB) or Diaminobenzidine (DAB); a colorimetric reaction appears; this is all the more intense when there are human or animal immunoglobulins fixed on the antigens present in the wells;
optionally, a new incubate is done for between 10 and 30 minutes at a temperature between 18 and 20° C., preferably protected from light; the reaction is then stopped very preferably in an acid solution;
the optical density of each well of the microtiter plates is then read, preferably using a spectrophotometer; the reading is preferably carried out at 450 nm with a correction alpha at 620 nm or at 650 nm; these optical densities are then preferably recorded in software.

According to a specific algorithm, the OD values of the patient's serum (human or animal) of all the specific markers defining his immunological profile are held up against the distribution of a population of patients suffering from a chronic autoimmune pathology versus the distribution of a population of healthy controls. These population distributions depend on a Mann and Whitney U test with a probability associated with a Mann and Whitney U test (P value) threshold of 0.01, on a distribution by percentiles.

It is thus possible to detect whether the human being or the animal to which the tested biological fluid belongs is affected by an autoimmune disease.

In addition, the method according to the invention also makes it possible to monitor the progression of the disease. The variation of the OD value for the sought antibody or antibodies makes it possible to verify the variation and the progression of the disease, defined by the P value of the Mann and Whitney U test:
If the P value is less than 0.01, defined by the Mann and Whitney U test, of the distribution of the patient population (human or animal) compared to the population distribution of healthy controls, then the level of circulating serum antibodies directed against a bacterial antigen and/or a neoantigen is significantly different from the level of circulating serum antibodies in a population of healthy controls.
If the P value is greater than 0.01, defined by the Mann and Whitney U test, of the distribution of the patient population (human or animal) compared to the population distribution of healthy controls, then the level of circulating serum antibodies directed against a bacterial antigen and/or a neoantigen is identical to the level of circulating serum antibodies in a population of healthy controls. The OD(s) obtained from the patient or animal are distributed by percentile according to the distribution chosen by the designed algorithm.

Assaying one or more specific antibodies also makes it possible to propose a treatment adapted as a function of the tested antibody or antibodies present in the sample.

According to one particular embodiment, the method according to the invention comprises at least the implementation of the following steps:
manufacturing at least one microtiter plate sensitized with the antigen(s) and/or the neoantigen(s) against which the antibodies, the presence of which is to be detected in the sample, are directed, or using at least one microtiter plate already sensitized with these antigens and/or neoantigen(s),
distributing the same volume of internal standards, sample and reagent blank on the plate,
adding the secondary antibody or antibodies coupled to an enzyme,
adding an enzyme substrate and a chromogen, waiting for the coloring of the wells,
stopping the coloring reaction,
reading the optical density of the wells using a spectrophotometer at an appropriate wavelength.

In particular, a particularly suitable embodiment of the invention is a method that comprises at least implementing the following steps:
manufacturing at least one microtiter plate sensitized with the antigen(s) and/or the neoantigen(s) against which the antibodies, the presence of which is to be detected in the sample, are directed, or using at least one microtiter plate already sensitized with these antigens and/or neoantigen(s),
diluting the sample to be tested and internal standards,
distributing, in duplicate on the plate, the same volume of diluted internal standards, diluted sample and reagent blank,
incubating,
washing,
adding the secondary antibody or antibodies coupled to peroxidase or alkaline phosphatase,
incubating,
washing,
adding a substrate and a chromogen,
stopping the reaction in acid solution
reading at 450 nm with a correction alpha at 650 or 620 nm.

The antibodies sought in the context of the method according to the invention, independent of the antigens against which they are directed, can be IgM (immunoglobulins of isotype M), IgA (immunoglobulins of isotype A), or IgG (immunoglobulins of isotype G), and in animals only, IgE (immunoglobulins of isotype E):
IgA antibodies: are the result of mucosal immune activation (intestinal, ENT, skin, bladder),
IgM antibodies: are the result of activation of the current immune system,
IgG antibodies: are the result of activation of the old immune system (memory immunity),
IgE antibodies (in animals only) are the result of the stimulation of mast cells, which release histamine, that is to say, they are the result of contact with allergenic antigens.

The method according to the invention is therefore carried out by immunoassay for the presence of antibodies in the sample, including at least:
one or more antibodies directed against at least one endobacterium, and
one or more antibodies directed against a product at the origin of or resulting from lipoperoxidation, and/or
one or more antibodies directed against a nitrated or nitrosylated product.

Thus, the method according to the invention is carried out by immunoassay for the presence of antibodies in the sample, including at least:
one or more antibodies directed against at least one enterobacterium, and one or more antibodies directed against a product at the origin of and/or resulting from lipoperoxidation, or
one or more antibodies directed against at least one enterobacterium, and one or more antibodies directed against a nitrated or nitrosylated product, or
one or more antibodies directed against at least one enterobacterium, and one or more antibodies directed against a product at the origin of and/or resulting from lipoperoxidation and one or more antibodies directed against a nitrated or nitrosylated product.

In fact, according to the invention, if these antibodies are present in the tested biological fluid sample, in particular in the tested serum and/or plasma, that is to say, if the test is positive for at least these antibodies, then this means that the person has a chronic autoimmune disease. Preferably, the method according to the invention comprises the immunoassay for the presence of at least one antibody directed against an enterobacterium (against a bacterial component of an enterobacterium) chosen from the following antibodies:
antibody or antibodies directed against the bacterium Pseudomonas putida,
antibody or antibodies directed against the bacterium Hafnia alvei,
antibody or antibodies directed against the bacterium Pseudomonas aeruginosa,
antibody or antibodies directed against the bacterium Pseudomonas pneumoniae,
antibody or antibodies directed against the bacterium Morganella morganii,
antibody or antibodies directed against the bacterium Proteus mirabilis
antibody or antibodies directed against the bacterium Citrobacter koserii.

Enterobacteria have a wall whose three-layer structure is specific to them. This wall is made up, from the outside to the inside, of: an outer membrane, a thin layer of peptidoglycan, and a periplasmic space that surrounds the cytoplasmic membrane. The outer membrane consists of a lipid double layer in which lipopolysaccharide molecules are included. The peptidoglycan forms a stiff, thinner and looser layer than in Gram-positive bacteria. It is composed of linear chains and polysaccharides linked together by peptides.

These bacteria are non-pathogenic and can be present on the mucous membranes. However, if they pass through the mucous membranes, they can be the source of chronic pathologies, in particular chronic autoimmune pathologies. Their transmucosal passage generates:
activation of the innate immune system,
activation of the adaptive immune system,
non-specific activation of self-reactive clones (superantigens),
direct toxicity (lipopolysaccharides, Pili type IV, exolysin, endotoxins)
activation of enzymatic systems: Inducible NO synthase and Indolamine 2,3-dioxygenase (IDO-1).

According to the invention, the presence in the tested biological fluid, in particular in the serum and/or in the plasma, of one or more antibodies directed against these bacterial components indicates mucosal hyperpermeability, which participates in the development of autoimmune diseases.

Preferably, the method according to the invention comprises immunoassay for the presence in the sample of:
IgG and/or IgM and/or IgA (and/or IgE for animals) directed against the bacterium Pseudomonas putida, and/or
IgG and/or IgM and/or IgA (and/or IgE for animals) directed against the bacterium Hafnia alvei, and/or
IgG and/or IgM and/or IgA (and/or IgE for animals) directed against the bacterium Pseudomonas aeruginosa, and/or
IgG and/or IgM and/or IgA (and/or IgE for animals) directed against the bacterium Pseudomonas pneumonae, and/or
IgG and/or IgM and/or IgA (and/or IgE for animals) directed against the bacterium Morganella morganii, and/or
IgG and/or IgM and/or IgA (and/or IgE for animals) directed against the bacterium Citrobacter koserii.

Preferably, the method according to the invention comprises immunoassay for the presence of at least one antibody directed against a product at the origin of or resulting from lipoperoxidation chosen from the following antibodies:
antibody or antibodies directed against palmitic acid,
antibody or antibodies directed against myristic acid,
antibody or antibodies directed against oleic acid,
antibody or antibodies directed against malondialdehyde
antibody or antibodies directed against azelaic acid,
antibody directed against a phospholipid.

NO is the source of many toxic radical species called reactive oxygen species (ROS). Among these ROSs, mention may in particular be made of lipid peroxides and their decomposition products, aldehydes, but also singlet oxygen and peroxynitrites. ROSs manifest their toxicity when they exceed cellular defense possibilities. These ROSs have an aggressive role. They attack phospholipid membranes, proteins and DNA. When they attack the phospholipid membranes, the radical attack can modify the lipid compounds constituting the cell and thus alter the messages coming from the environment toward the interior of the cell, causing a dysfunction that can be irreversible.

This lipoperoxidation is a cascade reaction that starts with the oxidation of the fatty acid chains constituting phospholipids, preferably unsaturated fatty acids having carbon-carbon double bonds. These unsaturated fatty acids ensure the fluidity of the membranes at best.

They become more unstable and more fragile.

Lipoperoxidation is made possible, on the one hand, by the existence of these double bonds of the polyunsaturated fatty acids, which facilitates the delocalization of the free electron, and, on the other hand, by the presence of molecular oxygen, which will easily pair one of its electrons with the delocalized free electron.

There are several types of lipoperoxidation:
a first, very active radical cascade, which will generate the production of free radicals,
the reaction of the O₂₋ superoxide radical with NO, which leads to the formation of very aggressive peroxynitrites that will cause the degradation of fatty acids into hydroperoxides, then into malondialdehyde (small molecule, extremely reactive with amino residues, and normally not present in the body).
another, milder type of lipoperoxidation causes, by hydrolysis, a breaking of the double bonds of the polyunsaturated fatty acids with the formation of diacids, one of the main compounds of which is azelaic acid.

The presence of lipoperoxidation products resulting from lipoperoxidation in humans or animals causes a disruption of cellular and membrane components resulting in the neoantigen formation, and consequently an immune and autoimmune activation at the origin of autoimmune disease.

Preferably, the method according to the invention comprises immunoassay for the presence in the sample of:
IgG and/or IgM and/or IgA (and/or IgE for animals) directed against myristic acid, and/or
IgG and/or IgM and/or IgA (and/or IgE for animals) directed against oleic acid, and/or
IgG and/or IgM and/or IgA (and/or IgE for animals) directed against malondialdehyde, and/or
IgG and/or IgM and/or IgA (and/or IgE for animals) directed against azelaic acid, and/or
IgG and/or IgM and/or IgA (and/or IgE for animals) directed against against a phospholipid.
IgG and/or IgM and/or IgA (and/or IgE for animals) directed against palmitic acid.

Preferably, the method according to the invention comprises the immunoassay for the presence of at least one antibody directed against a nitrated or nitrosylated product chosen from the following antibodies:
antibody or antibodies directed against NO-Cysteine,
antibody or antibodies directed against NO-asparagine,
antibody or antibodies directed against NO-histidine,
antibody or antibodies directed against NO-Tyrosine,
antibody or antibodies directed against NO₂-Tyrosine,
antibody or antibodies directed against NO-citrulline,
antibody or antibodies directed against NO-arginine,
antibody or antibodies directed against NO-Tryptophan,
antibody or antibodies directed against NO-methionine,
antibody or antibodies directed against NO-histamine,
antibody or antibodies directed against NO-phenylalanine,
antibody or antibodies directed against NO-proline.

NO (nitric oxide) has a single electron, which gives it very rapid reactivity. It depends on its ability to share its electron with other radicals or metals. In biological media, the synthesis of NO from L-arginine passes through an intermediate, hydroxy-L-arginine (HOA) under the action of NO synthase (NOS). Oxidation at the terminal nitrogen of the guanidine function leads to the formation of NO.

Two types of NOS are present in most vertebrates: so-called constitutive NOS (cNOS) and inducible NOS (iNOS).

Nitrated or nitrosylated products are neoantigens resulting from an overproduction of NO and/or peroxynitrite following the activation of inducible NO synthase, mainly in the cells of the innate immune system, by bacteria, viruses, pollutants, etc. The excess of NO and/or peroxynitrite results in their binding to endogenous proteins, which become immunogenic.

The endogenous proteins, the structure of which is thus modified, lead to immune activation and consequently autoimmune disease.

Preferably, the method according to the invention comprises the immunoassay for the presence in the sample of:
IgG and/or IgM and/or IgA (and/or IgE for animals) directed against NO-Cysteine, and/or
IgG and/or IgM and/or IgA (and/or IgE for animals) directed against NO-asparagine, and/or
IgG and/or IgM and/or IgA (and/or IgE for animals) directed against NO-histidine
IgG and/or IgM and/or IgA (and/or IgE for animals) directed against NO-Tyrosine, and/or
IgG and/or IgM and/or IgA (and/or IgE for animals) directed against NO₂-Tyrosine, and/or
IgG and/or IgM and/or IgA (and/or IgE for animals) directed against NO-citrulline, and/or
IgG and/or IgM and/or IgA (and/or IgE for animals) directed against NO-Tryptophan, and/or
IgG and/or IgM and/or IgA (and/or IgE for animals) directed against NO-methionine, and/or
IgG and/or IgM and/or IgA (and/or IgE for animals) directed against NO-histamine, and/or
IgG and/or IgM and/or IgA (and/or IgE for animals) directed against NO-phenylalanine, and/or
IgG and/or IgM and/or IgA (and/or IgE for animals) directed against NO-proline.

In addition (either at the same time or in a separate test) to the immunoassay for the presence in the sample of one or more antibodies directed against at least one bacterial component and of one or more antibodies directed against a tryptophan oxidation product, the method according to the invention can also comprise the immunoassay for the presence in the sample of one or more other antibodies. It may preferably be:
at least one antibody directed against a tryptophan oxidation product, and/or
at least one antibody directed against Mycobacterium tuberculosis.

Preferably, the method according to the invention comprises the immunoassay for the presence of at least one antibody directed against a tryptophan oxidation product chosen from the following antibodies:
antibody or antibodies directed against kynurenic acid,
antibody or antibodies directed against 3-hydroxykynurenine,
antibody or antibodies directed against quinolinic acid,
antibody or antibodies directed against quinaldic acid,
antibody or antibodies directed against xanthurenic acid,
antibody or antibodies directed against anthranilic acid,
antibody or antibodies directed against 3-hydroxyanthranilic acid,
antibody or antibodies directed against L-kynurenine.

L-tryptophan is metabolized into a large number of by-products: neurotransmitters, neurohormonals active in the nervous system. The best known are serotonin and melatonin by way of tryptophan hydroxylase.

It is an inducible enzymatic pathway. It is present in microglial neuronal cells and cells of the monocyte-macrophage lineage. There are many molecules that activate the IDO-1 pathway.

The main ones are the tumor necrosis factor (TNF)-a, interleukins IL-12 and IL-18, interferon-γ, microorganisms and their derivatives.

IDO-1 degrades L-tryptophan to N-formylkynurenine. A complex enzyme system then converts N-formylkynurenine to L-kynurenine. The latter is metabolized into different compounds by many enzymes. Kynureninase converts L-Kynurenine to anthranilic acid, which gives 3-hydroxy-anthranilic acid. This acid can then be converted into 3-hydroxyanthranilic acid. L-kynurenine is also degraded into 3-hydroxykynurenine by kynurenine 3-hydroxylase. The hydroxylated form is converted to 3-hydroxyanthranilic acid by a kynureninase. The latter compound is metabolized by 3-hydroxyanthranilic acid oxygenase to an intermediate product that originates in three distinct pathways. It gives either a picolinic acid by the picolinic carboxylase, or a quinolinic acid. L-kynurenine is converted to kynurenine by decarboxylases, or to kynurenic acid by kynurenine aminotransferase. This kynurenic acid is at the origin of the synthesis of quinaldic acid.

The IDO-1 pathway limits the pool of extracellular L-tryptophan, which promotes the replication of a large number of cells, and the tryptophan oxidation products, that is to say, the products resulting from the activation of the IDO pathway induce either cell apoptosis or immunotoxicity or cell and tissue protection. Their hyperproduction causes neoantigen formation, and consequently an autoimmune disease.

Preferably, the method according to the invention comprises the immunoassay for the presence in the sample of:
IgG and/or IgM and/or IgA (and/or IgE for animals) directed against kynurenic acid, and/or
IgG and/or IgM and/or IgA (and/or IgE for animals) directed against 3-hydroxykynurenine, and/or
IgG and/or IgM and/or IgA (and/or IgE for animals) directed against quinolinic acid, and/or
IgG and/or IgM and/or IgA (and/or IgE for animals) directed against quinaldic acid,
IgG and/or IgM and/or IgA (and/or IgE for animals) directed against xanthurenic acid, and/or
IgG and/or IgM and/or IgA (and/or IgE for animals) directed against anthranilic acid, and/or
IgG and/or IgM and/or IgA (and/or IgE for animals) directed against 3-hydroxyanthranilic acid
IgG and/or IgM and/or IgA (and/or IgE for animals) directed against L-kynurenine.

According to the invention, the presence in the biological fluid of at least one antibody directed against a tryptophan oxidation product, in particular IgA and/or Ig M and/or IgG (and/or IgE for animals), and the presence in the biological fluid of at least one antibody directed against an enterobacterium, in particular IgA and/or Ig M and/or IgG (and/or IgE for animals), necessarily means that the concerned person or animal is affected by an autoimmune disease.

The method according to the invention can also comprise the immunoassay for the presence of at least one antibody directed against Mycobacterium tuberculosis.

Mycobacterium tuberculosis is a Gram-positive pathogenic bacterium responsible for tuberculosis. It is also a factor in patients suffering from other diseases, such as people or animals suffering from certain autoimmune diseases. It does not produce toxins released uncontrolledly on fertile ground, but membrane components are released and have a toxic effect, and owe their pathogenic power to their ability to multiply. Lysis of the bacteria releases antigenic components that elicit an immune reaction inducing a state of hypersensitivity.

Their presence in humans or animals causes the release of membrane components (mycolic acid), some of which have toxic activity and activate the innate immune system by binding to specific receptors resulting in immune and autoimmune activation, and therefore in an autoimmune disease.

According to a particular embodiment, the method according to the invention is specifically intended for detecting or monitoring the progression of multiple sclerosis. In this case, the method is preferably carried out by immunoassay for the presence of antibodies in the sample, including at least:
one or more of the following antibodies:
   antibody or antibodies directed against the bacterium Pseudomonas putida,
   antibody or antibodies directed against the bacterium Hafnia alvei,
   antibody or antibodies directed against the bacterium Pseudomonas aeruginosa,
   antibody or antibodies directed against the bacterium Pseudomonas pneumoniae,
   antibody or antibodies directed against the bacterium Morganella morganii,
   antibody or antibodies directed against the bacterium Proteus mirabilis
   antibody or antibodies directed against the bacterium Citrobacter koserii
and one or more of the following antibodies:
   antibody or antibodies directed against palmitic acid,
   antibody or antibodies directed against oleic acid,
   antibody or antibodies directed against malondialdehyde
   antibody or antibodies directed against azelaic acid,
   antibody or antibodies directed against a phospholipid,
   antibody or antibodies directed against NO-Cysteine,
   antibody or antibodies directed against NO-asparagine,
   antibody or antibodies directed against NO-histidine,
   antibody or antibodies directed against NO-Tyrosine,
   antibody or antibodies directed against NO₂-Tyrosine,
   antibody or antibodies directed against NO-citrulline,
   antibody or antibodies directed against NO-arginine,
   antibody or antibodies directed against NO-Tryptophan,
   antibody or antibodies directed against NO-methionine,
   antibody or antibodies directed against NO-histamine,
   antibody or antibodies directed against NO-phenylalanine,
   antibody or antibodies directed against NO-proline.

The method in this case may also include immunoassay for the presence of one or more of the following antibodies:
antibody or antibodies directed against kynurenic acid,
antibody or antibodies directed against 3-hydroxykynurenine,
antibody or antibodies directed against quinolinic acid,
antibody or antibodies directed against quinaldic acid,
antibody or antibodies directed against xanthurenic acid,
antibody or antibodies directed against anthranilic acid,
antibody or antibodies directed against 3-hydroxyanthranilic acid,
antibody or antibodies directed against L-kynurenine.

Preferably, the detection of a high level of IgM for the antibodies sought according to the invention is characteristic of a relapsing-remitting form of multiple sclerosis, and the detection of a low level of IgM is characteristic of a progressive form or of a progressive relapsing-remitting form of multiple sclerosis. Likewise, preferably, the detection of a high level of Ig A for the antibodies sought according to the invention is characteristic of a progressive form or of a progressive relapsing-remitting form of multiple sclerosis.

Thus, in particular, according to a preferred embodiment, in the method according to the characterized invention, if the ratio IgM/IgA is greater than 1 (>1) for the detected antibodies, then there is (the patient is affected by) a relapsing-remitting form of multiple sclerosis. Likewise, according to a preferred embodiment, in the method according to the characterized invention, if the ratio IgA/IgM is greater than 1 (>1) for the detected antibodies, then there is (the patient is affected by) a progressive form or a progressive relapsing-remitting form of multiple sclerosis.

According to a particular embodiment, the method according to the invention is specifically intended for detecting or monitoring the progression of rheumatoid arthritis. In this case, the method is preferably carried out by immunoassay for the presence of antibodies in the sample, including at least:
one or more of the following antibodies:
   antibody or antibodies directed against the bacterium Pseudomonas putida,
   antibody or antibodies directed against the bacterium Hafnia alvei,
   antibody or antibodies directed against the bacterium Pseudomonas aeruginosa,
   antibody or antibodies directed against the bacterium Morganella morganii,
   antibody or antibodies directed against the bacterium Proteus mirabilis
   antibody or antibodies directed against the bacterium Citrobacter koserii
and one or more of the following antibodies:
   antibody or antibodies directed against palmitic acid,
   antibody or antibodies directed against myristic acid,
   antibody or antibodies directed against oleic acid,
   antibody or antibodies directed against malondialdehyde
   antibody or antibodies directed against azelaic acid,
   antibody or antibodies directed against a phospholipid
   antibody or antibodies directed against NO-Cysteine,
   antibody or antibodies directed against NO-asparagine,
   antibody or antibodies directed against NO-histidine,
   antibody or antibodies directed against NO-proline.

To implement the ex vivo method for detecting and/or monitoring the progression of a disease, the invention also relates to diagnostic kits.

In particular, the invention relates to a kit for use thereof in the detection or monitoring of the progression of a chronic autoimmune disease, in a sample of biological fluid, in particular in a sample of human or animal biological fluid comprising at least the antigen(s) against which the antibodies, the presence of which is to be detected in the sample, are directed, namely preferably one or more antibodies chosen from:
antibody or antibodies directed against the bacterium Pseudomonas putida,
antibody or antibodies directed against the bacterium Hafnia alvei,
antibody or antibodies directed against the bacterium Pseudomonas aeruginosa,
antibody or antibodies directed against the bacterium Pseudomonas pneumoniae,
antibody or antibodies directed against the bacterium Morganella morganii,
antibody or antibodies directed against the bacterium Proteus mirabilis
antibody or antibodies directed against the bacterium Citrobacter koserii
antibody or antibodies directed against palmitic acid,
antibody or antibodies directed against myristic acid,
antibody or antibodies directed against oleic acid,
antibody or antibodies directed against malondialdehyde
antibody or antibodies directed against azelaic acid,
antibody or antibodies directed against a phospholipid
antibody or antibodies directed against NO-Cysteine,
antibody or antibodies directed against NO-asparagine,
antibody or antibodies directed against NO-histidine,
antibody or antibodies directed against NO-Tyrosine,
antibody or antibodies directed against NO₂-Tyrosine,
antibody or antibodies directed against NO-citrulline,
antibody or antibodies directed against NO-arginine,
antibody or antibodies directed against NO-Tryptophan,
antibody or antibodies directed against NO-methionine,
antibody or antibodies directed against NO-histamine,
antibody or antibodies directed against NO-phenylalanine,
antibody or antibodies directed against NO-proline
antibody or antibodies directed against kynurenic acid,
antibody or antibodies directed against 3-hydroxykynurenine,
antibody or antibodies directed against quinolinic acid,
antibody or antibodies directed against quinaldic acid,
antibody or antibodies directed against xanthurenic acid,
antibody or antibodies directed against anthranilic acid,
antibody or antibodies directed against 3-hydroxyanthranilic acid
antibody or antibodies directed against L-kynurenine
antibody or antibodies directed against Mycobacterium tuberculosis.

Preferably, the kit according to the invention comprises at least:
the antigen(s) and/or neoantigen(s) against which the antibodies, the presence of which is to be detected in the sample, are directed, preferably coupled to a protein when they are not bacteria, and a microtiter plate intended to be sensitized with the antigen(s) and/or neoantigen(s) against which the antibodies, the presence of which is to be detected in the sample, are directed, or a microtiter plate already sensitized with the antigen(s) and/or neoantigen(s) against which the antibodies, the presence of which is to be detected in the sample, are directed, or a strip or stick already sensitized with the antigen(s) and/or neoantigen(s) against which the antibodies, the presence of which is to be detected in the sample, are directed, and
the standards making it possible to assess the quality of the test and the distribution of the control population, and/or
buffers and solutions suitable for performing an ELISA test, and/or
the secondary antibody or antibodies in solution, said secondary antibody or antibodies corresponding to the primary antibodies, the presence of which is to be detected in the sample and with the same isotypy, and/or
dilution and washing buffers, and/or
development buffers, and/or
a stop solution.

The invention is now illustrated by examples and results of implementing methods according to the invention.

For each example, the protocol was as follows:
collection of the patient's plasma
implementation of the method according to the invention
summary of the results.

The results make it possible to make the link between the distribution of the population affected by a chronic proliferative pathology compared to the distribution of a control population of the distributions of the patient populations.

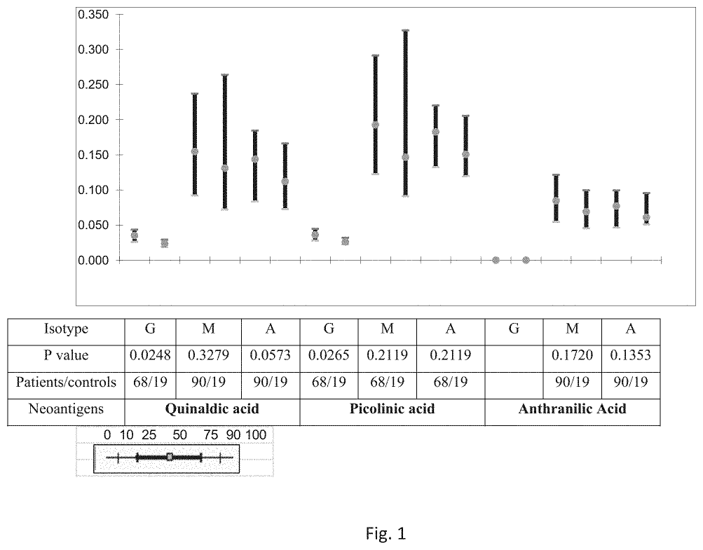

The results of the detection of circulating antibodies directed against quinaldic acid in IgA, IgM and IgG, picolinic acid in IgA, IgM and IgG and anthranilic acid in IgA, IgM and IgG, during the progression of multiple sclerosis are shown in FIG. 1, as a distribution of a population of multiple sclerosis patients; the midpoint represents 50% of the population.

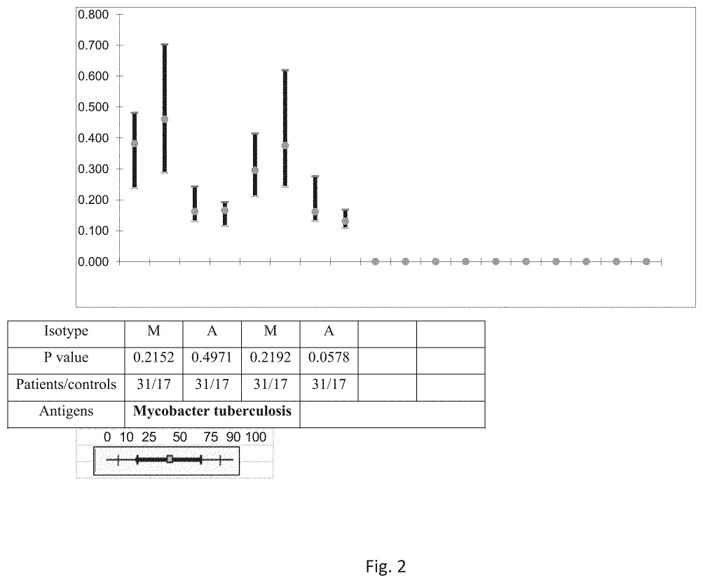

The results of the detection of circulating antibodies directed against Mycobacter tuberculosis in IgA, IgM and IgG, during the progression of multiple sclerosis are shown in FIG. 2, as a distribution of a population of multiple sclerosis patients; the midpoint represents 50% of the population.

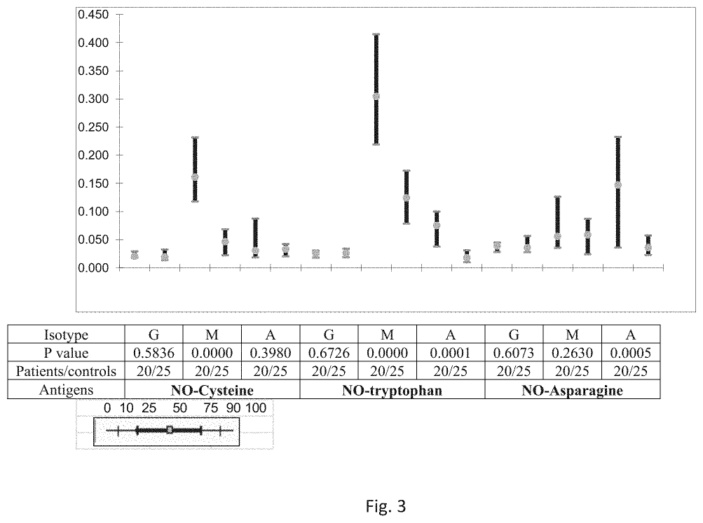

The results of the detection of circulating antibodies directed against NO-cysteine in IgA, IgM and IgG, NO-tryptophan in IgA, IgM and IgG and NO-asparagine in IgA, IgM and IgG, during the progression of progressive multiple sclerosis are shown in FIG. 3, as a distribution of a population of multiple sclerosis patients; the midpoint represents 50% of the population.

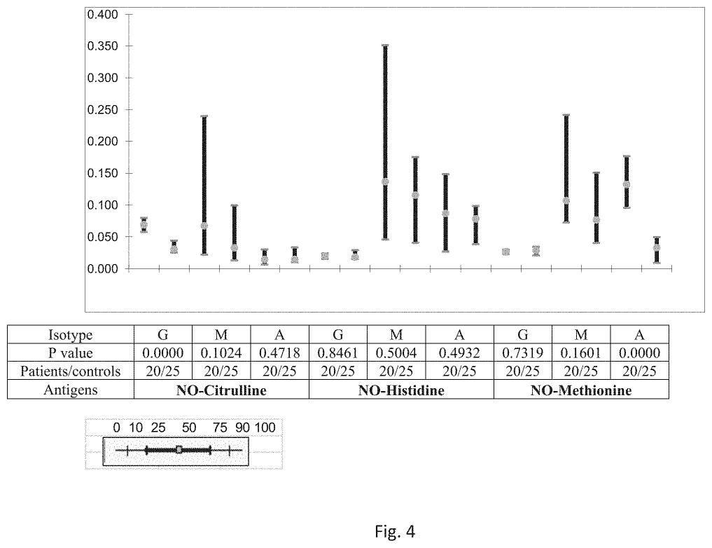

The results of the detection of circulating antibodies directed against NO-citrulline in IgA, IgM and IgG, NO-histidine in IgA, IgM and IgG and NO-methionine in IgA, IgM and IgG, during the progression of progressive multiple sclerosis are shown in FIG. 4, as a distribution of a population of multiple sclerosis patients; the midpoint represents 50% of the population.

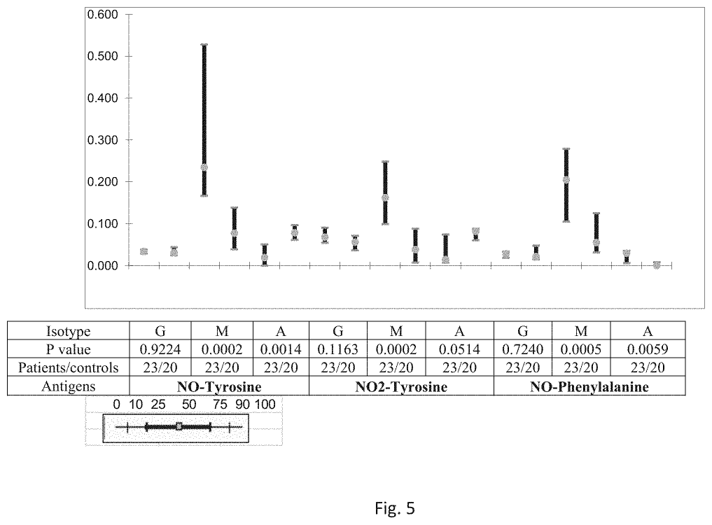

The results of the detection of circulating antibodies directed against NO-tyrosine in IgA, IgM and IgG, NO2-tyrosine in IgA, IgM and IgG and NO-phenylalanine in IgA, IgM and IgG, during the progression of relapsing-remitting multiple sclerosis are shown in FIG. 5, as a distribution of a population of multiple sclerosis patients; the midpoint represents 50% of the population.

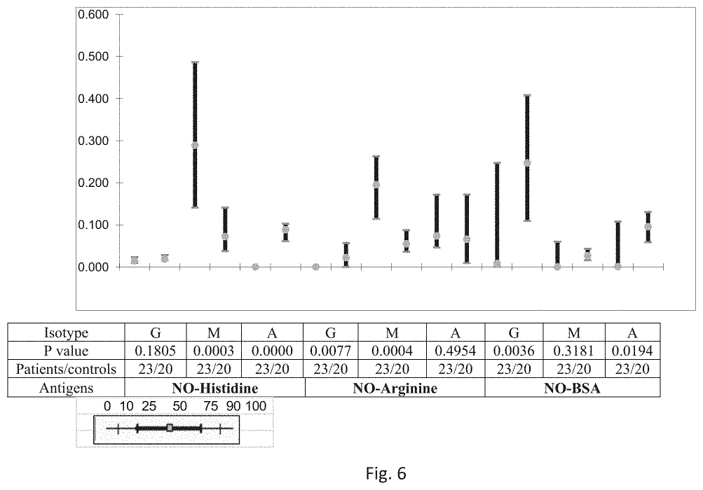

The results of the detection of circulating antibodies directed against NO-histidine in IgA, IgM and IgG, NO-arginine in IgA, IgM and IgG and NO-BSA in IgA, IgM and IgG, during the progression of relapsing-remitting multiple sclerosis are shown in FIG. 6, as a distribution of a population of multiple sclerosis patients; the midpoint represents 50% of the population.

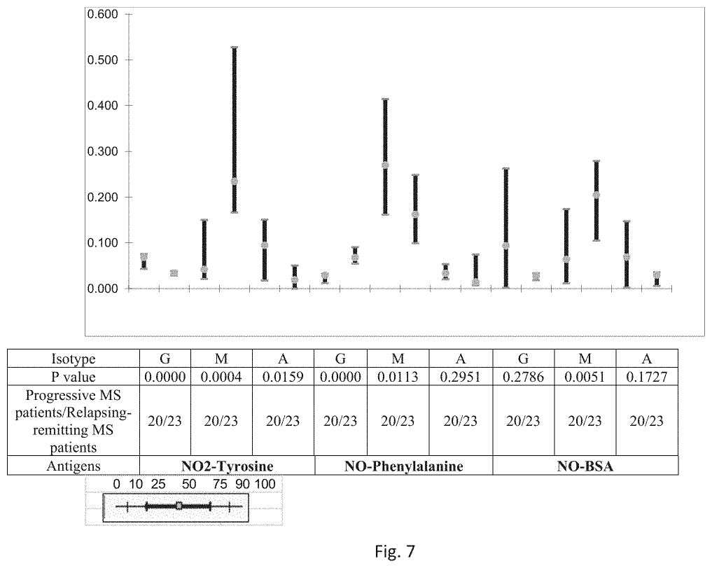

The results of the detection of circulating antibodies directed against NO2-tyrosine in IgA, IgM and IgG, NO-phenylalanine in IgA, IgM and IgG and NO-BSA in IgA, IgM and IgG, during the progression of multiple sclerosis are shown in FIG. 7, as a distribution of a population of patients with a progressive form of multiple sclerosis versus a relapsing-remitting form; the midpoint represents 50% of the population.

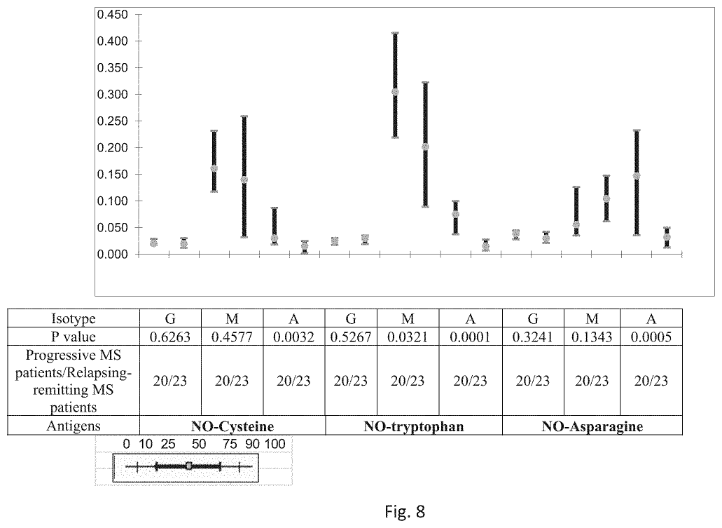

The results of the detection of circulating antibodies directed against NO-cysteine in IgA, IgM and IgG, NO-tryptophan in IgA, IgM and IgG and NO-asparagine in IgA, IgM and IgG, during the progression of multiple sclerosis are shown in FIG. 8, as a distribution of a population of patients with a progressive form of multiple sclerosis versus a relapsing-remitting form; the midpoint represents 50% of the population.

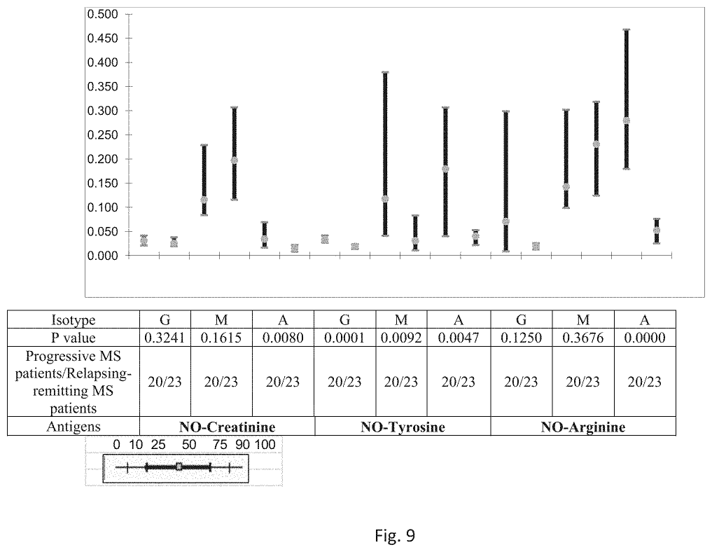

The results of the detection of circulating antibodies directed against NO-creatinine in IgA, IgM and IgG, NO-tyrosine in IgA, IgM and IgG and NO-arginine in IgA, IgM and IgG, during the progression of multiple sclerosis are shown in FIG. 9, as a distribution of a population of patients with a progressive form of multiple sclerosis versus a relapsing-remitting form; the midpoint represents 50% of the population.

1. An ex vivo method for detecting or monitoring the progression of a chronic autoimmune disease, in a sample of human or animal biological fluid, by immunoassay for the presence of antibodies in the sample, including at least:
one or more antibodies directed against at least one enterobacterium, chosen from the following antibodies:
antibody or antibodies directed against the bacterium Pseudomonas putida,
antibody or antibodies directed against the bacterium Hafnia alvei,
antibody or antibodies directed against the bacterium Pseudomonas aeruginosa,
antibody or antibodies directed against the bacterium Pseudomonas pneumonae,
antibody or antibodies directed against the bacterium Morganella morganii,
antibody or antibodies directed against the bacterium Proteus mirabilis
antibody or antibodies directed against the bacterium Citrobacter koserii, and
one or more antibodies directed against a product at the origin of or resulting from lipoperoxidation and/or one or more antibodies directed against a nitrated or nitrosylated product, wherein the antibody or antibodies directed against a lipoperoxidation product being chosen from the following antibodies:
antibody or antibodies directed against palmitic acid,
antibody or antibodies directed against myristic acid,
antibody or antibodies directed against oleic acid,
antibody or antibodies directed against malondialdehyde
antibody or antibodies directed against acetylcholine
antibody or antibodies directed against azelaic acid,
antibody or antibodies directed against a phospholipid,
and wherein the antibody or antibodies directed against the nitrated or nitrosylated product being chosen from the following antibodies:
antibody or antibodies directed against NO-Cysteine,
antibody or antibodies directed against NO-asparagine,
antibody or antibodies directed against NO-histidine,
antibody or antibodies directed against NO-Tyrosine,
antibody or antibodies directed against NO₂-Tyrosine,
antibody or antibodies directed against NO-citrulline,
antibody or antibodies directed against NO-arginine,
antibody or antibodies directed against NO-Tryptophan,
antibody or antibodies directed against NO-methionine,
antibody or antibodies directed against NO-histamine,
antibody or antibodies directed against NO-phenylalanine,
antibody or antibodies directed against NO-bovine serum albumin
antibody or antibodies directed against NO-proline.

2. The ex vivo method for detecting or monitoring the progression of a chronic autoimmune disease according to claim 1, characterized in that the sample is a sample of human or animal serum or plasma.

3. The ex vivo method for detecting or monitoring the progression of a chronic autoimmune disease according to claim 1, characterized in that the antibody or antibodies directed against at least one bacterial component are chosen from the following antibodies:
antibody or antibodies directed against the bacterium Pseudomonas putida,
antibody or antibodies directed against the bacterium Hafnia alvei,
antibody or antibodies directed against the bacterium Pseudomonas aeruginosa,
antibody or antibodies directed against the bacterium Pseudomonas pneumonae,
antibody or antibodies directed against the bacterium Morganella morganii,
antibody or antibodies directed against the bacterium Proteus mirabilis, and
antibody or antibodies directed against the bacterium Citrobacter koserii.

4. The ex vivo method for detecting or monitoring the progression of a chronic autoimmune disease according to claim 1, characterized in that the antibody or antibodies directed against a product at the origin of or resulting from lipoperoxidation are chosen from the following antibodies:
antibody or antibodies directed against palmitic acid,
antibody or antibodies directed against myristic acid,
antibody or antibodies directed against oleic acid,
antibody or antibodies directed against malondialdehyde,
antibody or antibodies directed against azelaic acid, and
antibody or antibodies directed against a phospholipid.

5. The ex vivo method for detecting or monitoring the progression of a chronic autoimmune disease according to claim 1, characterized in that the antibody or antibodies directed against a nitrated or nitrosylated product are chosen from the following antibodies:
antibody or antibodies directed against NO-Cysteine,
antibody or antibodies directed against NO-asparagine,
antibody or antibodies directed against NO-histidine,
antibody or antibodies directed against NO-Tyrosine,
antibody or antibodies directed against NO₂-Tyrosine,
antibody or antibodies directed against NO-citrulline,
antibody or antibodies directed against NO-arginine,
antibody or antibodies directed against NO-Tryptophan,
antibody or antibodies directed against NO-methionine,
antibody or antibodies directed against NO-histamine,
antibody or antibodies directed against NO-phenylalanine, and
antibody or antibodies directed against NO-proline.

6. The ex vivo method for detecting or monitoring the progression of a chronic autoimmune disease according to claim 1, characterized in that it also comprises the immunoassay in the sample for the presence of at least one antibody directed against a tryptophan oxidation product.

7. The ex vivo method for detecting or monitoring the progression of a chronic autoimmune disease according to claim 6, characterized in that the tryptophan oxidation products are chosen from the following antibodies:
antibody or antibodies directed against kynurenic acid,
antibody or antibodies directed against 3-hydroxykynurenine,
antibody or antibodies directed against quinolinic acid,
antibody or antibodies directed against quinaldic acid,
antibody or antibodies directed against xanthurenic acid,
antibody or antibodies directed against anthranilic acid,
antibody or antibodies directed against 3-hydroxyanthranilic acid, and
antibody or antibodies directed against L-kynurenic acid.

8. The ex vivo method for detecting or monitoring the progression of a chronic autoimmune disease according to claim , characterized in that it also comprises the immunoassay for the presence in the sample of at least one antibody directed against Mycobacterium tuberculosis.

9. The ex vivo method for detecting or monitoring the progression of a chronic autoimmune disease according to claim 1, characterized in that the antibodies are immunoglobulins A and/or immunoglobulins G and/or immunoglobulins M and/or, for animals only, immunoglobulins E.

10. The ex vivo method for detecting or monitoring the progression of a chronic autoimmune disease according to claim 1, characterized in that said disease is chosen from multiple sclerosis, rheumatoid arthritis and ankylosing spondylitis.

11. The ex vivo method for detecting or monitoring the progression of a chronic autoimmune disease according to claim 1, characterized in that said disease is multiple sclerosis and in that the method is carried out by immunoassay for the presence of antibodies in the sample, including at least:
one or more of the following antibodies:
antibody or antibodies directed against the bacterium Pseudomonas putida,
antibody or antibodies directed against the bacterium Hafnia alvei,
antibody or antibodies directed against the bacterium Pseudomonas aeruginosa,
antibody or antibodies directed against the bacterium Pseudomonas pneumoniae,
antibody or antibodies directed against the bacterium Morganella morganii,
antibody or antibodies directed against the bacterium Proteus mirabilis,
antibody or antibodies directed against the bacterium Citrobacter koserii;
and one or more of the following antibodies:
antibody or antibodies directed against palmitic acid,
antibody or antibodies directed against oleic acid,
antibody or antibodies directed against malondialdehyde
antibody or antibodies directed against azelaic acid,
antibody or antibodies directed against a phospholipid,
antibody or antibodies directed against NO-Cysteine,
antibody or antibodies directed against NO-asparagine,
antibody or antibodies directed against NO-histidine,
antibody or antibodies directed against NO-Tyrosine,
antibody or antibodies directed against NO₂-Tyrosine,
antibody or antibodies directed against NO-citrulline,
antibody or antibodies directed against NO-arginine,
antibody or antibodies directed against NO-Tryptophan,
antibody or antibodies directed against NO-methionine,
antibody or antibodies directed against NO-histamine,
antibody or antibodies directed against NO-phenylalanine,
antibody or antibodies directed against NO-proline.

12. The ex vivo method for detecting or monitoring the progression of a chronic autoimmune disease according to the preceding claim, characterized in that the method also comprises the immunoassay for the presence of one or more of the following antibodies:
antibody or antibodies directed against kynurenic acid,
antibody or antibodies directed against 3-hydroxykynurenine,
antibody or antibodies directed against quinolinic acid,
antibody or antibodies directed against quinaldic acid,
antibody or antibodies directed against xanthurenic acid,
antibody or antibodies directed against anthranilic acid,
antibody or antibodies directed against 3-hydroxyanthranilic acid, and
antibody or antibodies directed against L-kynurenic acid.

13. The ex vivo method for detecting or monitoring the progression of a chronic autoimmune disease according to claim 11, characterized in that a ratio IgM/IgA>1 for the detected antibodies is characteristic of a relapsing-remitting form of multiple sclerosis.

14. The ex vivo method for detecting or monitoring the progression of a chronic autoimmune disease according to claim 11, characterized in that a ratio IgA/IgM>1 for the detected antibodies is characteristic of a progressive form or of a progressive relapsing-remitting form of multiple sclerosis.

15. The ex vivo method for detecting or monitoring the progression of a chronic autoimmune disease according to claim 1, characterized in that said disease is rheumatoid arthritis and in that the method is carried out by immunoassay for the presence of antibodies in the sample, including at least:
one or more of the following antibodies:
antibody or antibodies directed against the bacterium Pseudomonas putida,
antibody or antibodies directed against the bacterium Hafnia alvei,
antibody or antibodies directed against the bacterium Pseudomonas aeruginosa,
antibody or antibodies directed against the bacterium Morganella morganii,
antibody or antibodies directed against the bacterium Proteus mirabilis,
antibody or antibodies directed against the bacterium Citrobacter koserii;
and one or more of the following antibodies:
antibody or antibodies directed against palmitic acid,
antibody or antibodies directed against myristic acid,
antibody or antibodies directed against oleic acid,
antibody or antibodies directed against malondialdehyde
antibody or antibodies directed against azelaic acid,
antibody or antibodies directed against a phospholipid
antibody or antibodies directed against NO-Cysteine,
antibody or antibodies directed against NO-asparagine,
antibody or antibodies directed against NO-histidine,
antibody or antibodies directed against NO-proline.

16. The ex vivo method for detecting or monitoring the progression of a chronic autoimmune disease according to claim 1, characterized in that the immunoassay is carried out by implementing an immuno-enzymatic method.

17. The ex vivo method for detecting or monitoring the progression of a chronic autoimmune disease according to claim 1, characterized in that it comprises at least the implementation of the following steps:
manufacturing at least one microtiter plate sensitized with the antigen(s) and/or the neo-antigen(s) against which the antibodies, the presence of which is to be detected in the sample, are directed, or using at least one microtiter plate already sensitized with these antigens and/or neo-antigens,
distributing the same volume of internal standards, sample and reagent blank on the plate,
adding the secondary antibody or antibodies coupled to an enzyme,
adding an enzyme substrate and a chromogen, waiting for the coloring of the wells
stopping the coloring reaction, and
reading the optical density of the wells using a spectrophotometer at an appropriate wavelength.

18. The ex vivo method for detecting or monitoring the progression of a chronic autoimmune disease according to the preceding claim, characterized in that it comprises at least the implementation of the following steps:
manufacturing at least one microtiter plate sensitized with the antigen(s) and/or the neoantigen(s) against which the antibodies, the presence of which is to be detected in the sample, are directed, or using at least one microtiter plate already sensitized with these antigens and/or neoantigens,
diluting the sample to be tested and internal standards,
distributing, in duplicate on the plate, the same volume of diluted internal standards, diluted sample and reagent blank,
incubating,
washing,
adding the secondary antibody or antibodies coupled to peroxidase or alkaline phosphatase,
incubating,
washing,
adding a substrate and a chromogen,
stopping the reaction in acid solution, and
reading at 450 nm with a correction alpha at 620 nm.

19. A kit for use in a method for detecting or monitoring the progression of a chronic autoimmune disease according to claim 1, characterized in that it comprises at least the neoantigen(s) against which the antibodies, the presence of which is to be detected in the sample, are directed.

20. The kit according to claim 19, characterized in that it comprises at least:
a microtiter plate sensitized with the antigen(s) and/or the neoantigen(s) against which the antibodies, the presence of which is to be detected in the sample, are directed,
buffers and solutions, and
the secondary antibody or antibodies in solution, secondary antibodies corresponding to the antibodies whereof the presence is to be detected in the sample.
